(19) United States
(12) Patent Application Publication
(10) Pub. No.: US 2020/0005940 A1
(43) Pub. Date: Jan. 2, 2020

(54) SYSTEM AND METHOD FOR GENERATING A CARE SERVICES COMBINATION FOR A USER

(71) Applicant: KONINKLIJKE PHILIPS N.V., Eindhoven (NL)

(72) Inventors: Cliff Johannes Robert Hubertina LASCHET, Gulpen (NL); Jorn OP DEN BUIJS, Eindhoven (NL); Steffen Clarence PAUWS, Eindhoven (NL)

(21) Appl. No.: 16/173,619

(22) Filed: Oct. 29, 2018

Related U.S. Application Data
(60) Provisional application No. 62/584,414, filed on Nov. 10, 2017.

(51) Int. Cl. G16H 50/20; G06N 20/20; G16H 10/60; G06K 9/62

(52) U.S. Cl. CPC G16H 50/20; G06K 9/6256; G16H 10/60; G06N 20/20

(57) ABSTRACT

The present system is configured to generate an ensemble prediction model to provide a care services combination for a user. The ensemble prediction model is configured to predict the effectiveness of individual care services for users. The ensemble prediction model accounts for effects of feature combinations on outcomes for the users. The present system is configured such that output from the ensemble prediction model is used during a single agent search to determine optimal combinations of services that minimize the risk of emergency re-hospitalization and/or other negative patient outcomes.

### BACKGROUND

### 1. Field

The present disclosure pertains to a system and method for generating a care services combination for a user by generating a prediction model which predicts an impact of any combination of care services for the user and performing a single agent search using predicted impact as a heuristic function to determine the care services combination.

### 2. Description of the Related Art

A wide variety of post-acute care medical services are available to patients to aid recovery, and computer systems are often utilized to generate recommendations of such medical services to patients. Such recommendations are traditionally standardized based on the acute care received, not tailored for the patient, and not determined based on possible combinatorial effects with other services. Moreover, given that the patient records relied upon by such computer systems to generate recommendations typically do not express all (or close to all) possible combinations of patient record data (e.g., due to practical limitations), the traditional statistical prediction technologies utilized by such computer systems often produce excessively complex models (e.g., with too many parameters relative to the number of observations). As such, typical recommendation models utilized by such computer systems frequently face random error or noise instead of the underlying relationship or other overfitting issues, often resulting in less-than-desirable recommendations as well as a waste of computer resources in generate such recommendations.

### SUMMARY

Accordingly, one or more aspects of the present disclosure relate to a system configured to generate a care services combination for a user by generating a prediction model which predicts an impact of any combination of care services for the user and performing a single agent search using predicted impact as a heuristic function to determine the care services combination. The system comprises one or more hardware processors configured by machine readable instructions, and/or other components. The one or more hardware processors are configured to obtain historical health information for a patient population. The historical health information indicates patient-related features. The patient-related features comprise demographics of patients of the patient population, physiological conditions of the patients, care services received by the patients, and corresponding outcomes for the patients. The one or more hardware processors are configured to initialize a set of feature combinations. Each feature combination of the set of feature combinations (i) is predictive of at least one of the corresponding outcomes and (ii) comprises two or more of the patient-related features of the historical health information. The one or more hardware processors are configured to generate a prediction model comprising a predetermined number of groups of feature combinations by performing the following operations: (A) randomly grouping feature combinations of the set of feature combinations into one or more groups of feature combinations; (B) with respect to each group of the one or more groups, selecting feature combinations in the group that are more predictive of at least one of the corresponding outcomes relative to other feature combinations in the group (note that various methods could be used to select feature combinations, for example, pruning the non-predictive combinations, selecting only a predetermined number of most predictive feature combinations, etc.); (C) re-initializing the set of feature combinations such that the set of feature combinations include the selected feature combinations and does not include one or more other feature combinations relative to which at least one of the selected feature combinations are more predictive (e.g., such that operation (C) includes re-initializing the set of feature combinations to only include those feature combinations that were selected in operation (B) and any other feature combinations that were not selected in operation (B) are discarded); and (D) re-performing operation (A) and, based on a determination that the re-performance of operation (A) results in more than the predetermined number of groups of feature combinations, re-performing operations (B), (C), and (D). The one or more hardware processors are configured to obtain health information for the user. The health information for the user is related to demographics of the user, physiological conditions of the user, and care received by the user. The one or more hardware processors are configured to generate a care services combination for the user based on the prediction model and the health information for the user. The care services combination comprises one or more of the care services received by the patients of the patient population. In some embodiments, the care services combination is generated via a single agent search and/or by other methods.

Another aspect of the present disclosure relates to a method for generating a care services combination for a user by generating a prediction model which predicts an impact of any combination of care services for the user and performing a single agent search using predicted impact as a heuristic function to determine the care services combination. The method is performed with a generation system. The system comprises one or more hardware processors configured by machine readable instructions, and/or other components. The method comprising obtaining, with the one or more hardware processors, historical health information for a patient population. The historical health information indicates patient-related features. The patient-related features comprise demographics of patients of the patient population, physiological conditions of the patients, care services received by the patients, and corresponding outcomes for the patients. The method comprises initializing, with the one or more hardware processors, a set of feature combinations. Each feature combination of the set of feature combinations (i) is predictive of at least one of the corresponding outcomes and (ii) comprises two or more of the patient-related features of the historical health information. The method comprises generating, with the one or more hardware processors, a prediction model comprising a predetermined number of groups of feature combinations by performing the following operations: (A) randomly grouping feature combinations of the set of feature combinations into one or more groups of feature combinations; (B) with respect to each group of the one or more groups, selecting feature combinations in the group that are more predictive of at least one of the corresponding outcomes relative to other feature combinations in the group (note that various methods could be used to select feature combinations, for example, pruning the non-predictive combinations, selecting only a predetermined number of most predictive feature combinations, etc.); (C) re-initializing the set of feature combinations such that the set of feature combinations include the selected feature combinations and does not include one or more other feature combinations relative to which at least one of the selected feature combinations are more predictive (e.g., such that operation (C) includes re-initializing the set of feature combinations to only include those feature combinations that were selected in operation (B) and any other feature combinations that were not selected in operation (B) are discarded); and (D) re-performing operation (A) and, based on a determination that the re-performance of operation (A) results in more than the predetermined number of groups of feature combinations, re-performing operations (B), (C), and (D). The method comprises obtaining, with the one or more hardware processors, health information for the user. The health information for the user is related to demographics of the user, physiological conditions of the user, and care received by the user. The method comprises generating, with the one or more hardware processors, a care services combination for the user based on the prediction model and the health information for the user. The care services combination comprises one or more of the care services received by the patients of the patient population. In some embodiments, the care services combination is generated via a single agent search and/or by other methods.

Still another aspect of present disclosure relates to a system for generating a care services combination for a user by generating a prediction model which predicts an impact of any combination of care services for the user and performing a single agent search using predicted impact as a heuristic function to determine the care services combination. The system comprises means for obtaining historical health information for a patient population. The historical health information indicates patient-related features. The patient-related features comprise demographics of patients of the patient population, physiological conditions of the patients, care services received by the patients, and corresponding outcomes for the patients. The system comprises means for initializing a set of feature combinations. Each feature combination of the set of feature combinations (i) is predictive of at least one of the corresponding outcomes and (ii) comprises two or more of the patient-related features of the historical health information. The system comprises means for generating a prediction model comprising a predetermined number of groups of feature combinations by performing the following operations: (A) randomly grouping feature combinations of the set of feature combinations into one or more groups of feature combinations; (B) with respect to each group of the one or more groups, selecting feature combinations in the group that are more predictive of at least one of the corresponding outcomes relative to other feature combinations in the group (note that various methods could be used to select feature combinations, for example, pruning the non-predictive combinations, selecting only a predetermined number of most predictive feature combinations, etc.); (C) re-initializing the set of feature combinations such that the set of feature combinations include the selected feature combinations and does not include one or more other feature combinations relative to which at least one of the selected feature combinations are more predictive (e.g., such that operation (C) includes re-initializing the set of feature combinations to only include those feature combinations that were selected in operation (B) and any other feature combinations that were not selected in operation (B) are discarded); and (D) re-performing operation (A) and, based on a determination that the re-performance of operation (A) results in more than the predetermined number of groups of feature combinations, re-performing operations (B), (C), and (D). The system comprises means for obtaining health information for the user. The health information for the user being related to demographics of the user, physiological conditions of the user, and care received by the user. The system comprises means for generating a care services combination for the user based on the prediction model and the health information for the user. The care services combination comprises one or more of the care services received by the patients of the patient population. In some embodiments, the care services combination is generated via a single agent search and/or by other methods.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates a system configured to generate a care services combination for a user.

FIG. 2 illustrates interactions between care services, and interactions between a patient-related feature and several care services.

FIG. 3 illustrates a number of services received by patients from an example dataset, and the frequency with which a particular number of services were received.

FIG. 4 illustrates operations performed by a feature component, a model component, and/or other components of the system.

FIG. 5 schematically illustrates operations performed by system.

FIG. 6 illustrates a single agent search tree structure.

FIG. 7 illustrates a method for generating a care services combination for a user.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

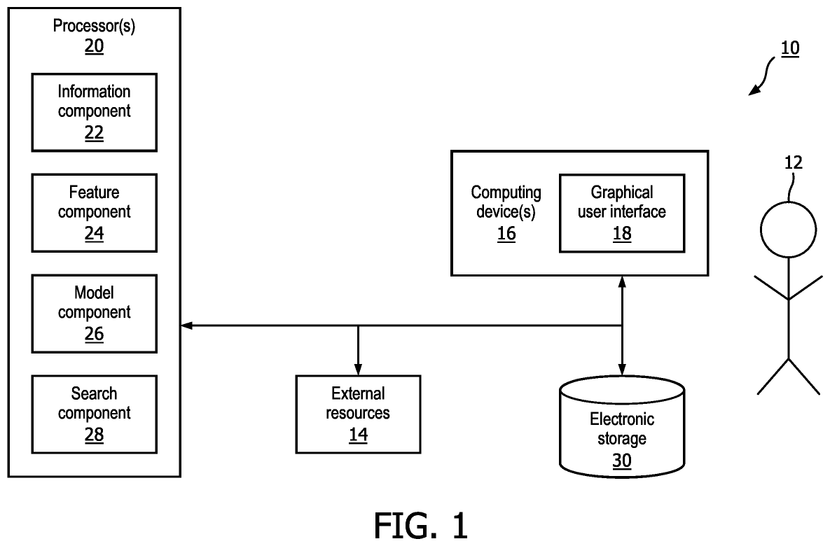

FIG. 1 is a schematic illustration of a system 10 configured to generate a care services combination for a user 12. A care services combination may be and/or include one or more care services for user 12 after acute care of user 12 is completed and/or for user 12 at other times. A care services combination may be and/or include a schedule and and/or information related to a schedule for corresponding services. For example, individual care services in a care services combination may be related to one or more of physical therapy, nutrition advice, exercise, weight loss coaching, medication management, addiction counseling and/or other treatment, mental health services, sleep monitoring, vital signs and/or other physiological monitoring, personal emergency response services, fall prevention programs, continuous positive airway pressure (CPAP) and/or other sleep apnea treatments, biofeedback, and/or other care services. The care services combination (e.g., activities plus schedule) may be configured to enhance patient outcomes (e.g., survival rates, quality of life, risk of hospital readmission, etc.), while simultaneously reducing costs, reducing and/or preventing emergency care, and/or providing other advantages. Unlike prior art systems, the care services of the care services combination generated by system 10 have a collaborative effect and enhance patient outcomes at least because system 10 determines care services for the combination based on individual patient-related features (e.g., demographic characteristics, physiological conditions of the patients, care services received by the patients, corresponding outcomes for the patients, and other features described below) when the combination is generated, and the effects of various care services in combination.

In prior art systems, available empirical data indicating treatment outcomes is not used effectively to determine which post-acute care services to recommend to a patient. Instead, care services are recommended based on past treatments and outcomes only for that patient, generally accepted treatment guidelines, and/or subjective opinions. As a result, relevant present features (e.g., demographic characteristics, physiological conditions, etc.) of individual patients may not be considered, and/or interactions between these features and possible care services, and their effect on treatment efficacy might be overlooked by prior art systems. This often leads prior art systems to recommend a set of post-acute care services that are not tailored to an individual patient, and/or to recommend an ineffective combination of post care services because of unexpected effects produced by combining features.

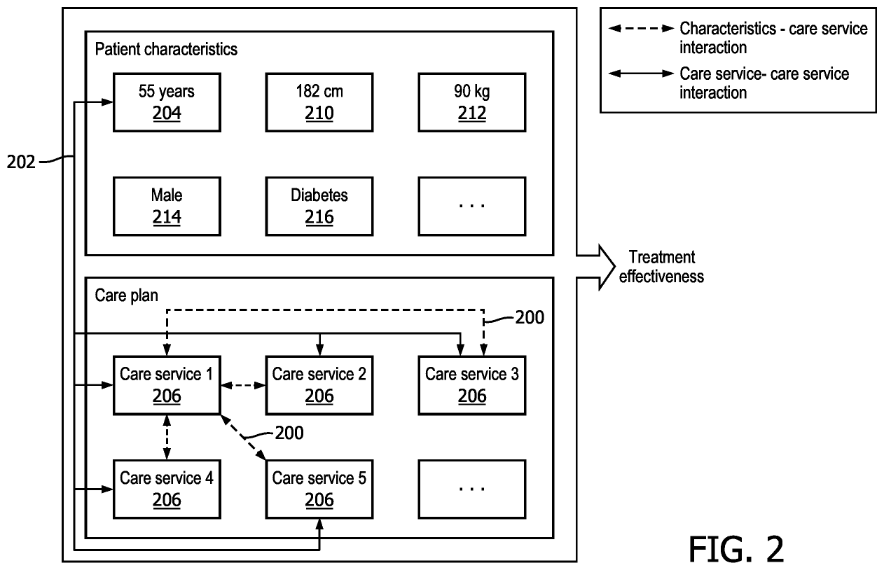

Combinations of care services often have a larger collaborative effect than the sum of their individual elements. Interactions between possible care services and/or patient-related features influence the overall treatment effectiveness of post-acute care. System 10 is configured to determine care services combinations based not only on present features of individual patients, but also the interactions between patient-related features and possible care services, and the interactions between multiple possible care services. For example, FIG. 2 illustrates interactions 200 (dotted lines) between possible care services, and interactions 202 (solid lines) between a patient-related feature (e.g., an age demographic characteristic) 204 with several care services 206 (care service 1-5). System 10 (FIG. 1) is configured such that interactions between care services 206 and/or with patient features (e.g., demographic characteristics 204, 210, 212, 214, physiological conditions such as diabetes 216) influence the generation of a care services combination for a patient (e.g., user 12 shown in FIG. 1).

Returning to FIG. 1, system 10 is configured to determine care services combinations based on both sets of interactions (e.g., between care services, between care services and patient-related features) and/or other information. System 10 is configured to automatically generate a combination of care services (the care services combination) configured to reduce and/or eliminate the risk of future undesirable health outcomes (e.g., emergency care comprising unplanned general practitioner visits and/or hospital appointments, etc.), treatment costs, and/or other outcomes. In some embodiments, system 10 comprises one or more of external resources 14, a computing device 16, a processor 20, electronic storage 30, and/or other components.

External resources 14 include sources of information and/or other resources. For example, external resources 14 may include heath and/or other information. The health information may be health information related to user 12, historical health information related to a population of patients, and/or other health information. In some embodiments, the population of patients includes patients similar to user 12. In some embodiments, the health information indicates patient-related features, features related to user 12, and/or other features. The patient-related features, the features related to user 12, and/or other features comprise demographics of patients of the patient population and/or user 12, physiological conditions of the patients and/or user 12, care services (e.g., acute care treatments, post-acute care services, etc.) received by the patients and/or user 12, corresponding outcomes for the patients and/or user 12, and/or other features. In some embodiments, external resources 14 include sources of information such as databases, websites, etc.; external entities participating with system 10 (e.g., a medical records system of a health care provider that stores medical history information for populations of patients), one or more servers outside of system 10, and/or other sources of information. In some embodiments, external resources 14 include components that facilitate communication of information such as a network (e.g., the internet), electronic storage, equipment related to Wi-Fi technology, equipment related to Bluetooth® technology, data entry devices, sensors, scanners, and/or other resources. External resources 14 may be configured to communicate with processor 20, computing devices 16, electronic storage 30, and/or other components of system 10 via wired and/or wireless connections, via a network (e.g., a local area network and/or the internet), via cellular technology, via Wi-Fi technology, and/or via other resources. In some embodiments, some or all of the functionality attributed herein to external resources 14 may be provided by resources included in system 10.

Computing devices 16 are configured to provide interfaces between user 12, caregivers (e.g., doctors, nurses, friends, family members, medical administrators, medical staff members, medical technicians, researchers, etc.), and/or other users, and system 10. In some embodiments, individual computing devices 16 are and/or are included in desktop computers, laptop computers, tablet computers, smartphones, and/or other computing devices associated with individual users 12, caregivers, and/or other users. In some embodiments, individual computing devices 16 are, and/or are included in hospital and/or other medical facility computing devices; equipment used in hospitals, doctor's offices, and/or other medical facilities to monitor patients 12; test equipment; equipment for treating patients; data entry equipment; and/or other devices. Computing devices 16 are configured to provide information to and/or receive information from caregivers, user 12, and/or other users. For example, computing devices 16 may be configured to present a graphical user interface 18 to users, caregivers, etc. to facilitate entry and/or selection of heath information, a predetermined number of groups of feature combinations (described below) and/or other information. In some embodiments, graphical user interface 18 is configured to display the care services combination to user 12, a caregiver, and/or other users. In some embodiments, graphical user interface 18 includes a plurality of separate interfaces associated with computing devices 16, processor 20, and/or other components of system 10; multiple views and/or fields configured to convey information to and/or receive information from caregivers, users 12, and/or other users; and/or other interfaces.

In some embodiments, computing devices 16 are configured to provide graphical user interface 18, processing capabilities, databases, electronic storage, and/or other resources to system 10. As such, computing devices 16 may include processors 20, electronic storage 30, external resources 14, and/or other components of system 10. In some embodiments, computing devices 16 are connected to a network (e.g., the internet).

In some embodiments, computing devices 16 do not include processors 20, electronic storage 30, external resources 14, and/or other components of system 10, but instead communicate with these components via the network. The connection to the network may be wireless or wired. For example, processor 20 may be located in a remote server and may wirelessly cause display of graphical user interface 18 to a caregiver and/or user 12 on a computing device 16 associated with the caregiver and/or user 12.

As described above, in some embodiments, an individual computing device 16 is a laptop, a personal computer, a smartphone, a tablet computer, and/or other computing devices. Examples of interface devices suitable for inclusion in an individual computing device 16 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that an individual computing device 16 includes a removable storage interface. In this example, information may be loaded into a computing device 16 from removable storage (e.g., a smart card, a flash drive, a removable disk) that enables the caregivers, user 12, and/or other users to customize the implementation of computing devices 16 and/or system 10. Other exemplary input devices and techniques adapted for use with computing devices 16 include, but are not limited to, an RS-232 port, RF link, an IR link, a modem (telephone, cable, etc.) and/or other devices.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, one or more computing devices 16 associated with user 12 and/or caregivers, devices that are part of external resources 14, electronic storage 30, and/or other devices.)

In some embodiments, processor 20, external resources 14, computing devices 16, electronic storage 30, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 14, computing devices 16, electronic storage 30, and/or other components according to a client/server architecture, a peer-to-peer architecture, and/or other architectures.

As shown in FIG. 1, processor 20 is configured via machine-readable instructions to execute one or more computer program components. The computer program components may comprise software programs and/or algorithms coded and/or otherwise embedded in processor 20, for example. The one or more computer program components comprise one or more of an information component 22, a feature component 24, a model component 26, a search component 28, and/or other components. Processor 20 may be configured to execute components 22, 24, 26, and/or 28 by software; hardware; firmware; some combination of software, hardware, and/or firmware; and/or other mechanisms for configuring processing capabilities on processor 20.

It should be appreciated that although components 22, 24, 26, and 28 are illustrated in FIG. 1 as being co-located within a single processing unit, in embodiments in which processor 20 comprises multiple processing units, one or more of components 22, 24, 26, and/or 28 may be located remotely from the other components. The description of the functionality provided by the different components 22, 24, 26, and/or 28 described below is for illustrative purposes, and is not intended to be limiting, as any of components 22, 24, 26 and/or 28 may provide more or less functionality than is described. For example, one or more of components 22, 24, 26, and/or 28 may be eliminated, and some or all of its functionality may be provided by other components 22, 24, 26, and/or 28. As another example, processor 20 may be configured to execute one or more additional components that may perform some or all of the functionality attributed below to one of components 22, 24, 26, and/or 28.

Information component 22 is configured to obtain historical health information for a patient population. In some embodiments, the historical health information comprises demographic information indicating demographics associated with patients, vital signs information indicating vital signs associated with patients, medical condition information indicating medical conditions experienced by patients, treatment information indicating treatments received by patients, information indicating post treatment services received by patients, outcome information indicating health and/or other outcomes for patients, and/or other historical health information. The historical health information indicates patient-related features and/or other information. The patient-related features comprise demographics of patients of the patient population, physiological conditions of the patients, care services received by the patients, corresponding outcomes for the patients, payment information related to the patients, International Statistical Classification of Diseases and Related Health Problems codes (ICD9 codes) for the patients, and/or other patient-related features. In some embodiments, care services include treatments received by the patients during acute care, post-hospitalization and/or post-acute care services received by the patients, and/or other care services. In some embodiments, the outcomes for the patients indicate information related to one or more of patient mortality, risk of readmission, treatment costs, and/or other outcomes.

By way of a non-limiting example, in some embodiments the historical health information may comprise demographic features (e.g., gender, ethnicity, age, etc.) associated with demographics of patients, vital signs features (e.g., heart rate, temperature, respiration rate, etc.) associated with vital signs associated with patients, medical condition features (e.g., a disease type, symptoms, behaviors, etc.) associated with medical conditions experienced by patients, treatment features (e.g., length of treatment, length of stay in a medical facility, medications, interventions, etc.) associated with treatments received by patients, outcome features (e.g., discharge date, prognosis, readmission date, cost, etc.) associated with health outcomes for patients, and/or other feature related information. It should be noted that the example features described above are not intended to be limiting. An uncountable number of possible features exist and those listed above are a small subset of examples.

Information component 22 is also configured to obtain health information for user 12. The health information for user 12 corresponds to the historical health information for the patient population. For example, the health information for user 12 is related to demographics of user 12, physiological conditions of user 12, care received by user 12, and/or other information.

In some embodiments, obtaining the historical health information, the health information for user 12, and/or other information includes electronically importing the information (e.g., from one or more databases included in external resources 14), facilitating entry and/or selection of the information (e.g., via computing devices 16), uploading and/or downloading information, receiving emails, texts, and/or other communications that include information, and/or other activities. For example, in some embodiments, the historical health information is stored in one or more databases (e.g., such as electronic databases included in external resources 14), and obtained by information component 22 from a database. In this example, information component 22 may obtain historical health information from medical records for a plurality of patients which include information such as acute treatments provided to patients, subsequent care services provided to patients, and corresponding health outcomes for the patients, and/or other information. In some embodiments, obtaining includes electronically importing only a portion and/or a subset of the historical health information (e.g., only information associated with specific features, etc.) from one or more databases. In some embodiments, the portion and/or subset may be determined at manufacture of system 10, determined by a user (e.g., a caregiver and/or user 12) via a user interface 18 of a computing system 16, and/or by other methods.

By way of a non-limiting example, historical health information obtained by information component 22 may be and/or include a publically available data set collected by the National Home and Hospice Care Survey (NHHCS) by the Centers for Disease Control and Prevention (CDC) group. This example information may be a nationally representative sample of U.S. home health and hospice agencies, providing information regarding their population of patients, staff and services. In some embodiments, as described above, information component 22 may be configured to obtain only a portion of this data. In this example, only data from non-hospice home care patients were used. This example dataset includes information for more than four thousand home care patients. Individual patients are described by roughly 300 features, including features related to demographic characteristics, financial information (e.g. insurance coverage, total sum billed for treatment, etc.), diagnoses, procedures, services, and/or other features. Individual patients in this dataset received one or more post-acute care services provided by an agency after being discharged from a hospital admission. The dataset also includes diagnoses of patients determined by medical staff and the procedures performed on patients (e.g. diabetes and hip replacement, respectively, as one possible example). The diagnoses and procedures are indicated in this example data set using ICD9 codes. The ICD9 codes in the NHHCS dataset provide links between medical conditions of the individual patients and the effect of post-acute services, which is relevant for a care services combination determination (e.g., as described below). Furthermore, this example dataset also indicates care plans (e.g., sets of post care services) provided to individual patients after hospital discharge. This example dataset includes 69 distinct services in total. Finally, the data set included information about the use of emergent care (e.g., emergency room visits, emergency outpatient and/or primary care physician's visit) in the last (e.g., most recent) 60 days of home care. This information was used as the outcome to be predicted by the predictive risk models (e.g., as described herein).

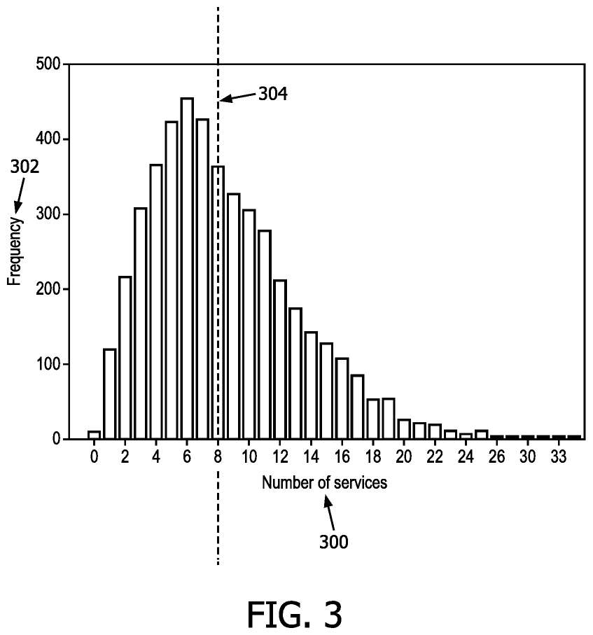

FIG. 3 illustrates the number of services 300 received by patients in the NHHCS example dataset and the frequency 302 with which a particular number of services was received. As shown in FIG. 3, on average 304, a patient in this dataset was provided with a combination of eight services. Table I lists examples of services provided to patients in the example NHHCS dataset for several example categories of services.

TABLE I
Care Services Examples

| Category | Examples |
|---|---|
| Assistive | Walk cane, motorized cart, bed |
| | communication, shower grab bars. |
| Medical | IV infusion pump, oxygen, apnea monitor, |
| | glucose monitor. |
| Agency provided | Training and explanation of device usage. |
| Personal care | Transport, meals on wheels, volunteers. |
| Therapy | Physical therapy, speech therapy, occupational |
| | therapy. |
| Counselling | Dietary counselling, ethical issues counselling, |
| | spiritual services. |
| Services provided | Bereavement services, medication management. |
| to family | |

Using this example dataset, for selection of eight services from a total of 69 possible services, there are about 8.4 billion possible combinations, (i.e., far too many for evaluation by a clinician in a clinically relevant time, or by prior art computerized service recommendation systems).

Returning to FIG. 1, feature component 24 is configured to initialize a set of feature combinations. Each feature combination of the set of feature combinations (i) is predictive of at least one of the corresponding outcomes and (ii) comprises two or more of the patient-related features of the historical health information. In some embodiments, the feature combinations comprise statistically significant predictive feature combinations of features from one or more of the demographics, the physiological conditions, the care services received by the patients, and/or other features on the outcomes for the patients. For example, in some embodiments, initializing a set of feature combinations comprises generating a set of individually significant interactions of features (e.g., age:cane), using tests of statistical significance (α=0.05 and/or other tests of significance), regarding an outcome variable (e.g., mortality, risk of readmission, cost, etc.). Any features directly related to a predicted outcome are not included. For example, if the predicted outcome is hospital readmission in the next 30 days, then a feature directly correlated to this is the patient's medical costs in the next 30 days. It will be clear for predictive modeling experts that such features (that are in fact measures of the future) may invalidate the predictive models. This is also known as (data) leakage. Continuing with the NHHCS example above, there are 272 individual features in the NHHCS dataset, of which 69 features represent possible care services for a care services combination. This means there are roughly 19000 interactions between services and/or other features tested for predictive power. Feature component 24 is configured such that feature combinations (e.g., interaction) for which no coefficient can be computed (e.g., due to a lack of variance in data) is discarded. In this example, this results in a set of 5449 distinct significant predictors, each of which describes an interaction comprising two services or one service and one patient characteristic.

Model component 26 is configured to generate a prediction model. The prediction model is generated using the historical health information, the feature combinations from feature component 24, and/or other information. Model component 26 is configured such that the prediction model quantifies the combined effects of possible care services and patient-related features on the outcome(s) of interest, taking into account the interaction effects of the possible care services and the patient-related features. Because the historical health information includes a finite amount of information (the NHHCS example described above includes roughly 5000 patient records and 300 features, which resulted in the determination and analysis of 19000 interactions), the limited number of records (for example) in the historical health information does not express all of the possible combinations of care services and/or patient-related feature values. (All of the possible combinations total more than the 19000 combinations for which NHHCS data was available). This means that all possible combinations of features cannot be quantified using the available data, and traditional statistical and/or machine learning techniques will result in overfitting.

Model component 26 is configured to utilize a suboptimal greedy approach similar to sequential backwards feature selection, with ensemble feature selection processes running in parallel where individual processes represent a group of selected features, and shuffling (e.g., randomly and/or using other methods) of remaining features between iterations and moving features from one group to another. This means that model component 26 is configured such that the prediction model comprises a predetermined number of groups of feature combinations. The prediction model is generated by performing operations including: grouping (e.g., randomly or otherwise) feature combinations of the set of feature combinations into one or more groups of feature combinations; with respect to each group of the one or more groups, selecting feature combinations in the group that are more predictive of at least one of the corresponding outcomes relative to other feature combinations in the group (note that various methods could be used to select feature combinations, for example, pruning the non-predictive combinations, selecting only a predetermined number of most predictive feature combinations, etc.); re-initializing the set of feature combinations such that the set of feature combinations include the selected feature combinations and does not include one or more other feature combinations relative to which at least one of the selected feature combinations are more predictive (e.g., such that the re-initialized set of feature combinations only includes those feature combinations that were selected in operation above and any other feature combinations that were not selected in the operation above are discarded); and re-performing the grouping of feature combinations into groups and, based on a determination that the number of groups results in more groups than the predetermined number of groups of feature combinations, re-performing the other operations. In some embodiments, grouping feature combinations comprises, with respect to each group of the one or more groups, generating an intermediate prediction model comprising the feature combinations of the group. In some embodiments, selecting more predictive feature combinations comprises, with respect to each group of the one or more groups, selecting feature combinations in the group that, as part of the generated intermediate prediction model, are more predictive of at least one of the corresponding outcomes relative to other feature combinations in the group.

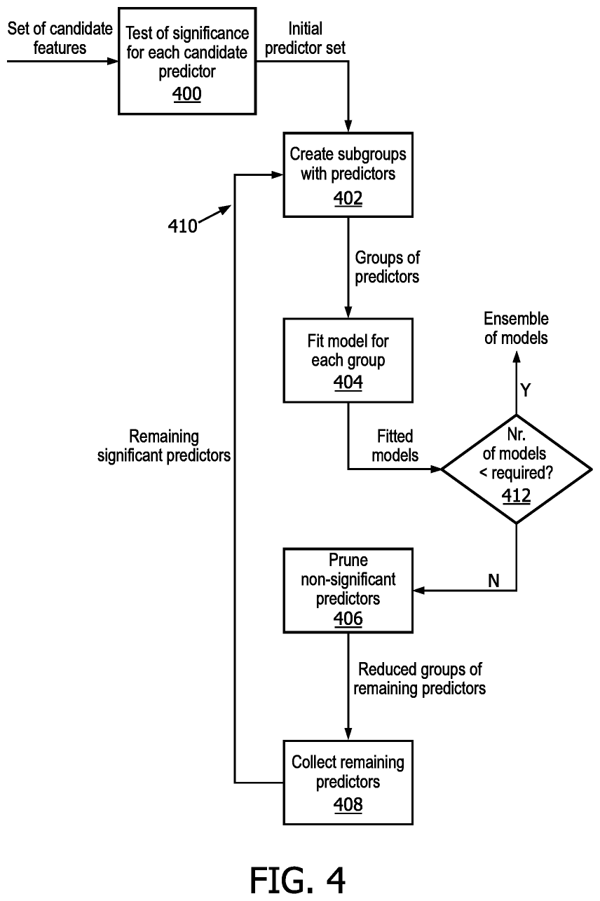

By way of a non-limiting example, operations performed by feature component 24, model component 26 (described above), and/or other components of system 10 are shown in FIG. 4. As shown in FIG. 4, a set of individually significant combinations of features (interaction predictors) is generated 400 (e.g., by feature component 24 shown in FIG. 1), using tests of significance (e.g., α=0.05), regarding an outcome variable (e.g., mortality, risk of readmission, costs, etc.). The set of (e.g., over five thousand continuing the NHHCS example above) feature combinations (predictors) is divided 402 (e.g., by model component 26 shown in FIG. 1) into a predefined number of groups. The predefined number of groups may be determined at manufacture of system 10, determined based on information in external resources 14 and/or electronic storage 30, determined based on information entered and/or selected via computing device(s) 16, and/or determined based on other information. Each group of feature combinations (predictors) is used to generate 404 an individual (e.g., logistic regression) model predicting the outcome variable. When combining these feature combinations (predictors) into an individual model, some of the feature combinations (predictors) may become insignificant. This phenomenon is also known as confounding. A confounder is a variable that influences both the outcome and another predictor. Individual models are then “pruned” 406 by removing any insignificant feature combinations (and/or conversely remaining significant feature combinations are selected). This results in a smaller group of feature combinations (predictors) which still have predictive power when combined with other predictors. Remaining feature combinations (predictors) are collected 408 from an individual model. This completes a single iteration. The process repeats 410 by shuffling and regrouping 402 the remaining feature combinations (predictors) from individual groups in the previous iteration into a smaller number of groups. Over one or more iterations, individual feature combinations (predictors) will be grouped with a high variety of other feature combinations (predictors).

In some embodiments, model component 26 (FIG. 1) is configured such that a number of groups for a given iteration is determined by dividing a number of remaining feature combinations by a number of feature combinations per group for that iteration. In such embodiments, the number of feature combinations per group for that iteration is determined by summing a number of feature combinations per group for an immediately previous iteration with a number of feature combinations not selected after the immediately previous iteration, and diving that sum by the number of groups in the immediately previous iteration. For example, the number of groups over iterations is determined as shown in Formula 1 shown below.

nᵢ=|features|/fᵢ

=fᵢ₋₁+(pᵢ/nᵢ₋₁) (1)

where the number of groups ηᵢ during iteration i is determined by dividing the number of remaining feature combinations by the number of feature combinations per group fᵢ during iteration i. It should be noted that these are feature combinations of two patient characteristics. Individual feature combinations are either a combination of a patient demographic with a specific care service (e.g. patient age with fall prevention), or a combination of two care services (e.g. fall prevention with personal emergency response services). The value of fᵢ is determined by summing the number of feature combinations per group of iteration i−1 and the number of pruned feature combinations ρᵢ, divided by the number of groups during the previous iteration. The initial values for ηᵢ and fᵢ are predefined (e.g., at manufacture, based on information from external resources 14 and/or electronic storage 30, based on information entered and/or selected via computing device(s) 16, etc.). This process ensures that as long as feature combinations (predictors) are being pruned (or conversely, remaining significant feature combinations are being selected), the number of groups will decrease over the iterations. By regrouping the remaining feature combinations into a smaller number of groups, individual models (e.g., groups) with an equal or larger number of feature combinations (predictors) are generated. Over the course of these iterations, the process converges to fewer models (groups) with larger sets of feature combinations (predictors) for individual models (groups), while also pruning any non-predictive and/or redundant feature combinations (predictors). The more feature combinations are pruned, the faster the process converges to fewer, but larger models (groups). The feature combination selection (grouping and regrouping) stops 412 when one model (group) remains, and/or when a predefined number (e.g., one or more) of models remain. Model component 26 is configured such that the resulting set of models is an ensemble model, where an output (e.g., a score, a ranking, etc.) assigned by the ensemble model is an aggregation (e.g., average and/or some other combination) over outputs returned by the individual models.

Continuing with the NHHCS dataset example above, model component 26 (FIG. 1), using the procedure illustrated in FIG. 4, generated an ensemble model comprising 15 (e.g., logistic regression) models. The ensemble model achieved an area under curve (AUC) of 76% (±2%) in 10-fold cross validation. The AUC values of the individual models (groups of feature combinations) varied from 69% (±3%) to 71% (±2%), which indicates that system 10 (FIG. 1) adds a significant amount of additional predictive performance relative to prior art systems.

Returning to FIG. 1, search component 28 is configured to generate one or more care services combinations for user 12. A care services combination is generated for user 12 based on the prediction model and the health information for user 12, and/or other information. The care services combination comprises one or more of the care services received by the patients of the patient population, and/or other care services. In some embodiments, search component 28 is configured to generate a care services combination that includes services similar to but different than the ones received by the patients in the patient population, for example. In some embodiments, the care services combination is generated via a single agent search. In some embodiments, individual care services in the care services combination comprise nodes in a node by node pathway from a root through an electronic tree structure, with each node of the tree structure comprising a possible service for the care services combination. The pathway through the tree structure may be selected based on output from the prediction model and/or other information.

As described above with respect to the NHHCS historical health information example, a patient may receive on average a care services recommendation of eight services. Since there are 69 options to choose from, this problem is computationally expensive. Assuming eight services can be combined into a care services combination, there are still more than 8×10\hat{ }9 distinct possible combinations of services. In a naïve exhaustive search for all possible care services combinations, which allows for different orderings, there will be more than 3.37×10\hat{ }14 leaf nodes. Thus, the time required for evaluating a combination of services for a specific patient is immense. Even when the evaluation of a single leaf node would take only 1 millisecond, a complete brute-force search would take more than 10,000 years to finish on a single-core machine.

In contrast to prior art systems, search component 28 is configured to perform a single agent search to generate one or more care service combinations for user 12. In some embodiments, search component 28 uses a Monte-Carlo Tree Search (MCTS). MCTS is a robust anytime search algorithm which does not require an admissible evaluation function. MCTS does require a utility function configured to assign a quality score to any state (e.g., combination of possible services). Model component 26 and search component 28 are configured such that the prediction model described above comprises this utility function for the MCTS. In the context of care services combination determinations, system 10 is configured such that this utility function (the prediction model) assesses how a given care services combination generated by search component 28 would affect a particular outcome for a specific patient (e.g., user 12). For example, system 10 is configured such that a combination of care services generated by search component 28 is ranked, scored, and/or otherwise evaluated using the prediction model described above (e.g., the ensemble of the predetermined number of intermediate models) to predict what effect that combination of services would have on a patient outcome of interest. Search component 28 is configured such that MCTS and/or other techniques combined with the prediction model are used to determine the care services combinations for users (e.g., user 12) and enhance target outcomes for the users (e.g., by ranking, scoring, and/or otherwise evaluating possible care services combinations determined by search component 28 relative to each other).

As described above, search component 28 is configured such that care services combination generation comprises a single-agent search and/or other operations. In some embodiments, search component 28 generates a currently selected possible combination of services for user 12. To change the configuration, search component 28 is configured to add services or to remove services from the currently selected possible combination of services. In some embodiments, search component 28 is configured such that this may be represented by a tree structure, for example. Search component 28 is configured such that a node in the search tree structure represents one of the 69 services found in the NHHCS dataset, for example. Service addition or subtraction actions are performed by transitioning from one node in the tree to a child or parent node, which changes the currently selected possible combination of services by adding or removing one service, respectively. A root node of the tree structure represents an empty care services combination. Using this tree structure, search component 28 is configured such that a care services combination is represented as a path through the tree, starting from the root node. Search component 28 is configured such that children of a node are defined such that repeats of already selected care services are not allowed. Search component 28 is configured such that the branching factor of node n_{d} is 69-d (using the NHHCS data for example), where d is the depth of the tree in which the node is situated. Search component 28 is configured such that the tree may still include duplicate care services combinations, where the only difference is the ordering of the services in the care services combination. Search component 28 is configured to assume that the order in which services are chosen to form a care plan has no effect on the efficacy of the care plan as a whole, as the services may be provided simultaneously to user 12, for example. In some embodiments, the size of the search tree is limited by allowing combinations of up to eight services (e.g., the average number of services patients were receiving in the NHHCS dataset). In some embodiments, the size of the search tree is limited by allowing combinations of up to ten services. In some embodiments, the size of the search tree is limited by allowing combinations of up to twenty services.

In some embodiments, search component 28 is configured such that a MCTS comprises four phases. For example, i) in a selection phase states (e.g., combinations of possible services) are encountered that are part of the search tree; ii) when a state is not in the tree, a simulation strategy chooses successor states until the end of the search (e.g., the roll out phase); iii) MCTS then expands the tree by adding the first state it encountered along its roll-out (e.g., the expansion phase); and iv) the result of the simulation is then back propagated to every node visited in the simulation up to the root node, updating node statistics accordingly for each node (e.g., the backpropagation phase). These four phases are repeated until a predetermined amount of time for the search expires. Search component 28 is configured such that a selection strategy is applied recursively from the root node of the tree, until an unknown position is encountered. This unknown position is not part of the search tree, yet. From the unknown position in the tree, a simulation is started (usually comprising performing random actions, e.g., adding care services randomly to the care services combination) until the bottom of the tree is reached (e.g., 8 care services have been selected). The evaluation score of the care package (e.g., the combination of 8 care services), provided by the predictive ensemble model, is backpropagated starting from the node added in the previous phase, to the root node. This ensures that the visited tree nodes more accurately resemble the potential outcome of interest for which the search is being performed.

In some embodiments, search component 28 is configured such that the MCTS returns a sequence of care services combinations instead of a single care services combination after the search has ended (e.g., because service selection is a single-agent search). In some embodiments, search component 28 is configured such that the roll-out phase of MCTS includes (e.g., randomly) selecting care services that have not yet been selected for a current possible care plan. In some embodiments, search component 28 is configured such that no actions automatically terminate the search. In such embodiments, search component 28 is configured such that a search ends in a terminal state responsive to no further actions being allowed, which is determined by search component 28 based on the limit of forming care plans of up to eight (for example) services described above. In some embodiments, search component 28 and/or model component 26 are configured such that possible care services combinations generated by search component 28 are ranked, scored, and/or otherwise evaluated based on mortality, risk of readmission (e.g., within a given time period), cost, and/or other patient outcomes, by the ensemble of models described above. The ranks, scores, and/or other evaluations returned by the prediction model are used by search component 28 such that the MCTS outputs one or more care services combinations for user 12 that decrease and/or eliminate negative outcomes for user 12.

In some embodiments, search component 28 is configured such that the MCTS comprises time-controlled MCTS and Progressive History/MAST (Move-Average Sampling Technique). In order to prioritize the selection of each service in a treatment package equally, search component 28 is configured such that the search is spread out across the full depth of the search tree. Time-controlled MCTS provides a way of doing this by dividing the allowed search time by the number of actions that may be performed. Time-controlled MCTS starts by performing a search for selecting the first service of the care plan, with an allowed search time of t=T/d where T is the total allowed search time and d is the number of services to select. The general idea of Progressive History (PH) is that actions that have shown to be successful in certain situations might be good actions in other similar situations as well. For care service selection, this means that services that are effective in general might be good candidates for selection when there is no clear winner among all the children of a node. To achieve this, PH stores for each action a corresponding relative history score in a table. MAST includes performing the best available action, according to the heuristic scores of the Progressive History table, with probability 1−ε. A random action is executed with a small probability ε (e.g., 0.1) to prevent the simulations from becoming too deterministic In some embodiments, search component 28 is configured such that the MCTS comprises time-controlled MCTS and Progressive History MAST because the quantity of possible care services combinations may be too large to traverse completely within reasonable time limits. With a large set of possible care services, a majority of MCTS search time may be spent on generating an upper part of the tree (e.g., evaluating the first few services included in a care services combination), while only a fraction of a lower portion of the tree (e.g., the last few services that may be included in a care services combination) is explored by Monte-Carlo simulations.

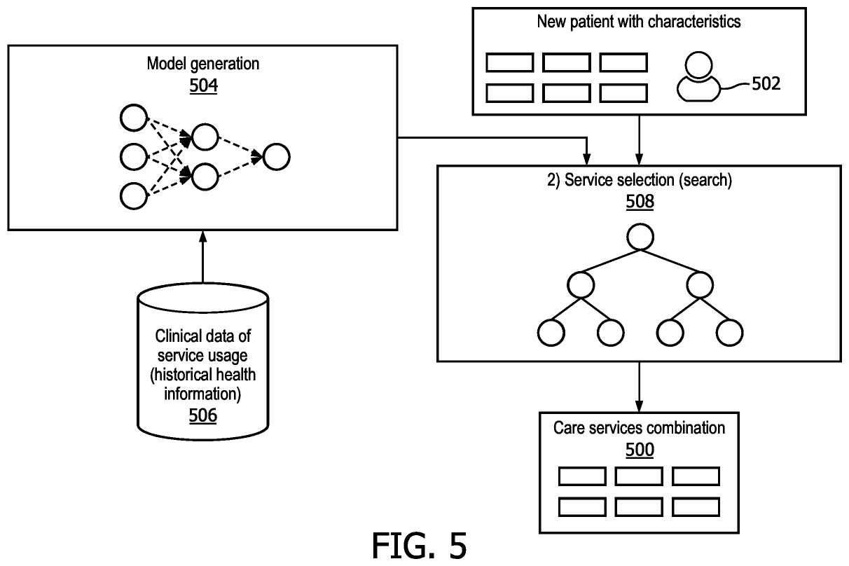

FIG. 5 schematically illustrates a summary and/or other indication of operations performed by system 10 (FIG. 1). As shown in FIG. 5, in order to predict the effectiveness of a care services combination 500 on a patient 502 (e.g., user 12 shown in FIG. 1), a prediction model 504 is generated (e.g., by model component 26 shown in FIG. 1) based on historical health information 506 (e.g., clinical data describing the service usage and outcome of a population of real patients and/or other information). As described above, prediction model 504 provides a representation of the interactions between multiple care services, and/or between care services and patient-related features (e.g., demographic characteristics, etc.). In some embodiments, prediction model 504 may also output a value for any possible care services combination indicating the effectiveness of the care services combination for a specific patient (e.g., user 12) and outcome of interest (e.g., risk of readmission). As described above, predictive model 504 is generated using historical health information 506, predictive feature combinations, and/or other information. In some embodiments, historical health information 506 describes historic patient demographic and/or physiological characteristics, medical service usage and outcomes, and/or other information as described herein (though any clinical dataset may be used to generate model 504 provided the data describes the effect of care services on some outcome of interest). As described above, model 504 comprises an ensemble model of individual (e.g., logistic regression) models. Model 504 may be used for the outcome prediction of new service combinations and new patients 502.

While prediction model 504 may evaluate a given care services combination, model 504 may not directly indicate which care service combination(s) would provide enhanced outcomes for a patient (e.g., user 12). This is because many possible combinations of care services are present in the prediction model, and the best combination of services requires a search of the features of the prediction model. The predictive model provides a prediction of the outcome of interest given a set of care services (e.g., a function from care services to predicted outcome, s1, s2, s3, . . . →o). The search can provide a(n) (close to) optimal combination of care services, given an evaluator for whatever outcome of interest (i.e. the predictive ensemble model in this case) for scoring nodes in the tree search. In other words, the search is a function from evaluator to combination of care services: e→s1, s2, s3, . . . . Hence, the predictive model cannot provide a combination of care services as output, but a search can use the predictive model to provide a combination of care services. As such, search 508 determines which services should be selected to form a care services combination. The large number of possible services to choose from, plus the fact that these services must be combined, make this a challenging single-agent search problem (e.g., as described above). In some embodiments, system 10 (FIG. 1) is configured such that the problem of service selection may be described in states representing care services combinations and actions representing individual care services that may be selected to be included in the care services combination. Predictive model 504 is used by system 10 as a utility function during search 508 to indicate the quality of proposed combinations of services given patient-related features (e.g., also as described above).

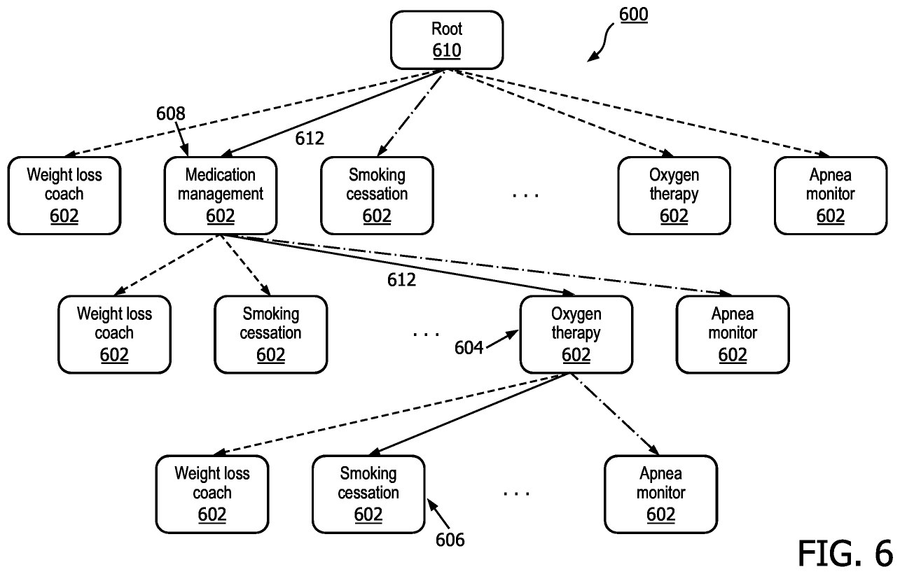

In some embodiments, as described above, search 508 is and/or includes a single agent search and/or other searches. In some embodiments, the single agent search is a Monte Carlo Tree Search and/or other searches. As shown in FIG. 6, the single agent search may be represented by a tree structure 600. A node 602 in the search tree structure 600 represents one of the care services in the historical health information (e.g., 506 in FIG. 5). Search 508 (FIG. 5) is configured to transition from one node 604 in tree structure 600 to a child 606 or parent node 608, which changes the care services combination by adding or removing exactly one service, respectively. The root node 610 represents an empty care services combination. Using this structure, a care services combination is represented as a path 612 through tree structure 600, starting from root node 610. System 10 is configured such that children of a node 602 are defined in a way that repeats of already chosen services are not permitted in a care services combination. However, tree structure 600 may still include duplicate care services combinations, where the only difference between care services combinations is the ordering of the services in the care services combinations. In some embodiments, system 10 assumes that the order in which services are chosen to form a care services combination has no effect on the efficacy of the care services combination as a whole, as the services will be provided simultaneously to the patient.

Returning to FIG. 1, electronic storage 30 comprises electronic storage media that electronically stores information. The electronic storage media of electronic storage 30 may comprise one or both of system storage that is provided integrally (i.e., substantially non-removable) with system 10 and/or removable storage that is removably and/or electronically connectable to system 10 via, for example, a port (e.g., a USB port, a firewire port, etc.) or a drive (e.g., a disk drive, etc.). Electronic storage 30 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), cloud storage, and/or other electronically readable storage media. Electronic storage 30 may store software algorithms, information determined by processor 20, information received via computing device(s) 16 and/or external computing systems (e.g., external resources 14), and/or other information that enables system 10 to function as described herein. Electronic storage 30 may be (in whole or in part) a separate component within system 10, or electronic storage 30 may be provided (in whole or in part) integrally with one or more other components of system 10 (e.g., processor 20, a computing device 16, a server that is part of system 10, and/or other components).

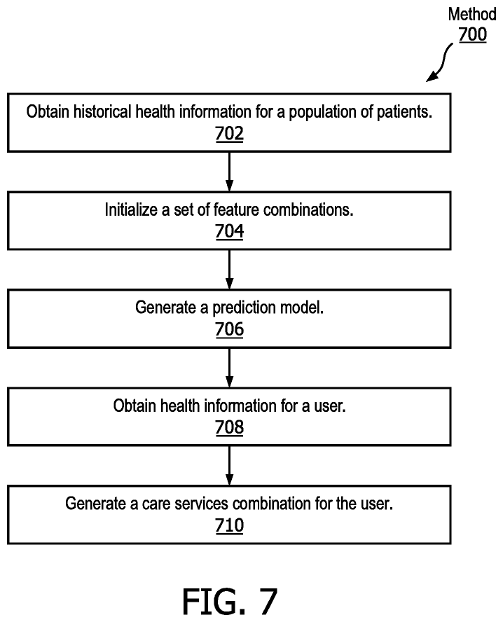

FIG. 7 illustrates method 700 for generating a care services combination for a user with a generation system by generating a prediction model which predicts an impact of any combination of care services for the user and performing a single agent search using predicted impact as a heuristic function to determine the care services combination. The system comprises one or more hardware processors configured by machine readable instructions, and/or other components. The one or more hardware processors are configured to execute computer program components. The computer program components comprise an information component, a feature component, a model component, a search component, and/or other components. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

In some embodiments, method 700 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 700 in response to instructions stored electronically on an electronic storage medium. The one or more processing devices may include one or more devices configured through hardware, firmware, and/or software to be specifically designed for execution of one or more of the operations of method 700.

At an operation 702, historical health information for a patient population is obtained. The historical health information indicates patient-related features and/or other information. The patient-related features comprise demographics of patients of the patient population, physiological conditions of the patients, care services received by the patients, corresponding outcomes for the patients, and/or other patient-related features. In some embodiments, operation 702 is performed by a processor component the same as or similar to information component 22 (shown in FIG. 1 and described herein).

At an operation 704, a set of feature combinations is initialized. Each feature combination of the set of feature combinations (i) is predictive of at least one of the corresponding outcomes and (ii) comprises two or more of the patient-related features of the historical health information. In some embodiments, the feature combinations comprise statistically significant predictive feature combinations of features from one or more of the demographics, the physiological conditions, or the care services received by the patients, on the outcomes for the patients. In some embodiments, operation 704 is performed by a processor component the same as or similar to feature component 24 (shown in FIG. 1 and described herein).

At an operation 706, a prediction model is generated. The prediction model comprises a predetermined number of groups of feature combinations. The prediction model is generated by performing the following operations: (A) randomly grouping feature combinations of the set of feature combinations into one or more groups of feature combinations; (B) with respect to each group of the one or more groups, selecting feature combinations in the group that are more predictive of at least one of the corresponding outcomes relative to other feature combinations in the group (note that various methods could be used to select feature combinations, for example, pruning the non-predictive combinations, selecting only a predetermined number of most predictive feature combinations, etc.); (C) re-initializing the set of feature combinations such that the set of feature combinations include the selected feature combinations and does not include one or more other feature combinations relative to which at least one of the selected feature combinations are more predictive (e.g., such that operation (C) includes re-initializing the set of feature combinations to only include those feature combinations that were selected in operation (B) and any other feature combinations that were not selected in operation (B) are discarded); and (D) re-performing operation (A) and, based on a determination that the re-performance of operation (A) results in more than the predetermined number of groups of feature combinations, re-performing operations (B), (C), and (D). In some embodiments, operation (A) comprises, with respect to each group of the one or more groups, generating an intermediate prediction model comprising the feature combinations of the group. In some embodiments, operation (B) comprises, with respect to each group of the one or more groups, selecting feature combinations in the group that, as part of the generated intermediate prediction model, are more predictive of at least one of the corresponding outcomes relative to other feature combinations in the group. In some embodiments, a number of groups for a given iteration are determined by dividing a number of remaining feature combinations by a number of feature combinations per group for that iteration. In such embodiments, the number of feature combinations per group for that iteration is determined by summing a number of feature combinations per group for an immediately previous iteration with a number of feature combinations not selected after the immediately previous iteration, and diving that sum by the number of groups in the immediately previous iteration. In some embodiments, operation 706 is performed by a processor component the same as or similar to model component 26 (shown in FIG. 1 and described herein).

At an operation 708, health information for the user is obtained. The health information for the user is related to demographics of the user, physiological conditions of the user, care received by the user, and/or other information. In some embodiments, operation 708 is performed by a processor component the same as or similar to information component 22 (shown in FIG. 1 and described herein).

At an operation 710, a care services combination is generated for the user. The care services combination is generated for the user based on the prediction model and the health information for the user, and/or other information. The care services combination comprises one or more of the care services received by the patients of the patient population, and/or other care services. In some embodiments, the care services combination is generated via a single agent search. In some embodiments, individual care services in the care services combination comprise nodes in a node by node pathway from a root through an electronic tree structure, with each node of the tree structure comprising a possible service for the care services combination. The pathway through the tree structure may be selected based on output from the prediction model and/or other information. In some embodiments, operation 710 is performed by a processor component the same as or similar to search component 28 (shown in FIG. 1 and described herein).

Experimental data produced using system 10 (FIG. 1) is described in Example 1 below. This example is not intended to be limiting.

### Example 1

Using the NHHCS dataset, system 10 was configured such that generation of care services combinations using MCTS was implemented in Java, while the ensemble prediction models were created in the R data analytics software package. A search time of 60 seconds was allowed and a combination of 8 services was the size of a given care services combination for a single patient. In this example, the MCTS configuration achieved 960 simulations per second on average (e.g., averaged over care services combination generations for an identical set of 100 distinct patients, stratified over risk deciles). In this example, the performance measure used during generation of the care services combinations for individual patients was average risk reduction. Risk reduction was defined based on an initial risk and a resulting risk, which both indicate a risk of emergency re-hospitalization, expressed as a percentage. The initial risk was assessed using a set of services and/or devices recommended originally by a clinician associated with the NHHCS dataset. The resulting risk was determined by calculating a risk score for the same patient when using the care services combination generated by MCTS. The difference between initial and resulting risk, expressed as percentage points, indicated the reduced risk per patient. The larger the risk reduction, the better MCTS (system 10) is performing.

Individual MCTS experiments showed that a large reduction in emergency care risk relative to prior art systems was possible using system 10. While the average reduced risk over all patients was 11.8 percentage points, the largest risk reductions were achieved in the highest risk deciles. The care services combination generation by system 10 reduced the risk of patients in the highest decile by 38.9 percentage points on average. The experiments generated a selection of exactly 8 services, as this was the average number of services a patient received in the NHHCS dataset. However, in actual practice, there is a large spread in the number of services received by various patients. System 10 was also used to investigate how the average risk reduction varied when the number of services in a care services combination was altered. The results are displayed in Table II.

TABLE II
Experimental Results

| No. Of | Risk decile | | | | | | | | | | |
|---|---|---|---|---|---|---|---|---|---|---|---|
| Services | 1st | 2nd | 3rd | 4th | 5th | 6th | 7th | 8th | 9th | 10th | All deciles |
| 1 | 0.66 | 1.61 | 2.71 | 3.47 | 4.68 | 7.05 | 8.67 | 12.85 | 13.43 | 27.02 | 8.21 ± 0.01 |
| 5 | 1.12 | 2.44 | 4.08 | 5.02 | 6.65 | 9.08 | 11.67 | 15.48 | 18.68 | 36.38 | 11.06 ± 0.015 |
| 8 | 1.27 | 2.72 | 4.49 | 5.46 | 7.21 | 9.68 | 12.49 | 16.39 | 20.32 | 38.86 | 11.89 ± 0.014 |
| 10 | 1.28 | 2.81 | 4.52 | 5.48 | 7.47 | 9.94 | 12.68 | 16.62 | 20.83 | 39.71 | 12.14 ± 0.019 |
| 15 | 1.32 | 2.94 | 4.75 | 5.79 | 7.69 | 10.14 | 12.91 | 16.97 | 21.53 | 40.55 | 12.46 ± 0.020 |
| 20 | 1.35 | 2.90 | 4.84 | 5.71 | 7.78 | 10.28 | 13.15 | 17.03 | 21.66 | 41.35 | 12.61 ± 0.003 |
| 30 | 1.36 | 3.01 | 4.80 | 5.90 | 7.84 | 9.67 | 13.06 | 17.15 | 21.93 | 40.96 | 12.57 ± 0.089 |

As shown in Table II, performance of system 10 relative to prior art systems steadily increases when larger combinations of services are included in the care services combinations. However, even when the care services combination comprises only one service, MCTS (system 10) still achieves a risk reduction of 8.21 percentage points. This means that a single post-acute service determined by system 10 reduces the risk of re-hospitalization significantly compared to prior art systems. As shown in Table II, far higher risk reductions are achieved for larger combinations of services, especially among the higher deciles.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system configured to generate a care services combination for a user by generating a prediction model which predicts an impact of any combination of care services for the user and performing a single agent search using predicted impact as a heuristic function to determine the care services combination, the system comprising one or more hardware processors configured by machine readable instructions to:
obtain historical health information for a patient population, the historical health information indicating patient-related features, the patient-related features comprising demographics of patients of the patient population, physiological conditions of the patients, care services received by the patients, and corresponding outcomes for the patients;
initialize a set of feature combinations, each feature combination of the set of feature combinations (i) being predictive of at least one of the corresponding outcomes and (ii) comprising two or more of the patient-related features of the historical health information;
generate a prediction model comprising a predetermined number of groups of feature combinations by performing the following operations:
(A) randomly grouping feature combinations of the set of feature combinations into one or more groups of feature combinations;
(B) with respect to each group of the one or more groups, selecting feature combinations in the group that are more predictive of at least one of the corresponding outcomes relative to other feature combinations in the group;
(C) re-initializing the set of feature combinations such that the set of feature combinations include the selected feature combinations and does not include one or more other feature combinations relative to which at least one of the selected feature combinations are more predictive; and
(D) re-performing operation (A) and, based on a determination that the re-performance of operation (A) results in more than the predetermined number of groups of feature combinations, re-performing operations (B), (C), and (D);
obtain health information for the user, the health information for the user being related to demographics of the user, physiological conditions of the user, and care received by the user; and
generate a care services combination for the user based on the prediction model and the health information for the user, the care services combination comprising one or more of the care services received by the patients of the patient population, the care services combination generated based on the prediction model via a single agent search of the one or more care services received by the patients of the patient population.

2. The system of claim 1, wherein the one or more hardware processors are configured such that:
operation (A) comprises, with respect to each group of the one or more groups, generating an intermediate prediction model comprising the feature combinations of the group, and
operation (B) comprises, with respect to each group of the one or more groups, selecting feature combinations in the group that, as part of the generated intermediate prediction model, are more predictive of at least one of the corresponding outcomes relative to other feature combinations in the group.

3. The system of claim 1, wherein the one or more hardware processors are configured such that individual care services in the care services combination comprise nodes in a node by node pathway from a root through an electronic tree structure, each node of the tree structure comprising a possible service for the care services combination, the pathway through the tree structure being selected based on output from the prediction model.

4. The system of claim 1, wherein the one or more hardware processors are configured such that the feature combinations comprise statistically significant predictive feature combinations of features from one or more of:
the demographics,
the physiological conditions, or
the care services received by the patients,
on the outcomes for the patients.

5. The system of claim 1, wherein the one or more hardware processors are configured such that a number of groups for a given iteration is determined by dividing a number of remaining feature combinations by a number of feature combinations per group for that iteration, and wherein the number of feature combinations per group for that iteration is determined by summing a number of feature combinations per group for an immediately previous iteration with a number of feature combinations not selected after the immediately previous iteration, and diving that sum by the number of groups in the immediately previous iteration.

6. A method for generating a care services combination for a user with a generation system by generating a prediction model which predicts an impact of any combination of care services for the user and performing a single agent search using predicted impact as a heuristic function to determine the care services combination, the system comprising one or more hardware processors configured by machine readable instructions, the method comprising:
obtaining, with the one or more hardware processors, historical health information for a patient population, the historical health information indicating patient-related features, the patient-related features comprising demographics of patients of the patient population, physiological conditions of the patients, care services received by the patients, and corresponding outcomes for the patients;
initializing, with the one or more hardware processors, a set of feature combinations, each feature combination of the set of feature combinations (i) being predictive of at least one of the corresponding outcomes and (ii) comprising two or more of the patient-related features of the historical health information;
generating, with the one or more hardware processors, a prediction model comprising a predetermined number of groups of feature combinations by performing the following operations:
(A) randomly grouping feature combinations of the set of feature combinations into one or more groups of feature combinations;
(B) with respect to each group of the one or more groups, selecting feature combinations in the group that are more predictive of at least one of the corresponding outcomes relative to other feature combinations in the group;
(C) re-initializing the set of feature combinations such that the set of feature combinations include the selected feature combinations and does not include one or more other feature combinations relative to which at least one of the selected feature combinations are more predictive; and
(D) re-performing operation (A) and, based on a determination that the re-performance of operation (A) results in more than the predetermined number of groups of feature combinations, re-performing operations (B), (C), and (D);
obtaining, with the one or more hardware processors, health information for the user, the health information for the user being related to demographics of the user, physiological conditions of the user, and care received by the user; and
generating, with the one or more hardware processors, a care services combination for the user based on the prediction model and the health information for the user, the care services combination comprising one or more of the care services received by the patients of the patient population, the care services combination generated based on the prediction model via a single agent search of the one or more care services received by the patients of the patient population.

7. The method of claim 6, wherein:
operation (A) comprises, with respect to each group of the one or more groups, generating an intermediate prediction model comprising the feature combinations of the group, and
operation (B) comprises, with respect to each group of the one or more groups, selecting feature combinations in the group that, as part of the generated intermediate prediction model, are more predictive of at least one of the corresponding outcomes relative to other feature combinations in the group.

8. The method of claim 6, wherein individual care services in the care services combination comprise nodes in a node by node pathway from a root through an electronic tree structure, each node of the tree structure comprising a possible service for the care services combination, the pathway through the tree structure being selected based on output from the prediction model.

9. The method of claim 6, wherein the feature combinations comprise statistically significant predictive feature combinations of features from one or more of:
the demographics,
the physiological conditions, or
the care services received by the patients,
on the outcomes for the patients.

10. The method of claim 6, wherein a number of groups for a given iteration is determined by dividing a number of remaining feature combinations by a number of feature combinations per group for that iteration, and wherein the number of feature combinations per group for that iteration is determined by summing a number of feature combinations per group for an immediately previous iteration with a number of feature combinations not selected after the immediately previous iteration, and diving that sum by the number of groups in the immediately previous iteration.

11. A system for generating a care services combination for a user by generating a prediction model which predicts an impact of any combination of care services for the user and performing a single agent search using predicted impact as a heuristic function to determine the care services combination, the system comprising:
means for obtaining historical health information for a patient population, the historical health information indicating patient-related features, the patient-related features comprising demographics of patients of the patient population, physiological conditions of the patients, care services received by the patients, and corresponding outcomes for the patients;
means for initializing a set of feature combinations, each feature combination of the set of feature combinations (i) being predictive of at least one of the corresponding outcomes and (ii) comprising two or more of the patient-related features of the historical health information;
means for generating a prediction model comprising a predetermined number of groups of feature combinations by performing the following operations:
(A) randomly grouping feature combinations of the set of feature combinations into one or more groups of feature combinations;
(B) with respect to each group of the one or more groups, selecting feature combinations in the group that are more predictive of at least one of the corresponding outcomes relative to other feature combinations in the group;
(C) re-initializing the set of feature combinations such that the set of feature combinations include the selected feature combinations and does not include one or more other feature combinations relative to which at least one of the selected feature combinations are more predictive; and
(D) re-performing operation (A) and, based on a determination that the re-performance of operation (A) results in more than the predetermined number of groups of feature combinations, re-performing operations (B), (C), and (D);
means for obtaining health information for the user, the health information for the user being related to demographics of the user, physiological conditions of the user, and care received by the user; and
means for generating a care services combination for the user based on the prediction model and the health information for the user, the care services combination comprising one or more of the care services received by the patients of the patient population, the care services combination generated based on the prediction model via a single agent search of the one or more care services received by the patients of the patient population.

12. The system of claim 11, wherein:
operation (A) comprises, with respect to each group of the one or more groups, generating an intermediate prediction model comprising the feature combinations of the group, and
operation (B) comprises, with respect to each group of the one or more groups, selecting feature combinations in the group that, as part of the generated intermediate prediction model, are more predictive of at least one of the corresponding outcomes relative to other feature combinations in the group.

13. The system of claim 11, wherein individual care services in the care services combination comprise nodes in a node by node pathway from a root through an electronic tree structure, each node of the tree structure comprising a possible service for the care services combination, the pathway through the tree structure being selected based on output from the prediction model.

14. The system of claim 11, wherein the feature combinations comprise statistically significant predictive feature combinations of features from one or more of:
the demographics,
the physiological conditions, or
the care services received by the patients,
on the outcomes for the patients.

15. The system of claim 11, wherein a number of groups for a given iteration is determined by dividing a number of remaining feature combinations by a number of feature combinations per group for that iteration, and wherein the number of feature combinations per group for that iteration is determined by summing a number of feature combinations per group for an immediately previous iteration with a number of feature combinations not selected after the immediately previous iteration, and diving that sum by the number of groups in the immediately previous iteration.
